SEQUENCE LISTING (2 sequences)

SEQ ID NO: 1 - DNA, 30 bp, Artificial Sequence
  Primer
agcgttttagcgccggcgccggtgcaggac

SEQ ID NO: 2 - DNA, 38 bp, Artificial Sequence
  Primer
cgccggcgctaaaacgctttcctggaggagctgcggcc

(19) United States
(12) Patent Application Publication
(10) Pub. No.: US 2009/0263866 A1
(43) Pub. Date: Oct. 22, 2009

(54) Industrial-scale Serum-free Production of Recombinant Factor VII in Mammalian Cells

(75) Inventors: GILES WILSON, Copenhagen O (DK); Ida Molgaard Knudsen, Vaerlose (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk HealthCare A/G, Zurich (CH)

(21) Appl. No.: 12/495,964

(22) Filed: Jul. 1, 2009

Related U.S. Application Data
(63) Continuation of application No. 10/394,086, filed on Mar. 21, 2003, now abandoned.
Application No. 10/394,086 is a continuation of application No. PCT/DK01/00634, filed on Oct. 2, 2001.
(60) Provisional application No. 60/238,944, filed on Oct. 10, 2000.
(60) Provisional application No. 60/271,581, filed on Feb. 26, 2001.
(60) Provisional application No. 60/276,322, filed on Mar. 16, 2001.

(30) Foreign Application Priority Data
Oct. 2, 2000 (DK) PA 2000 01456
Feb. 16, 2001 (DK) PA 2001 00262
Mar. 14, 2001 (DK) PA 2001 00430
May 14, 2001 (DK) PA 2001 00751

(51) Int. Cl. C12P 21/02

(52) U.S. Cl. 435/70.4

(57) ABSTRACT

The invention provides a method for industrial-scale production of FVII polypeptides in mammalian cell culture free of animal-derived components.

### CROSS-REFERENCE TO RELATED APPLICATIONS

This application is a continuation of U.S. application Ser. No. 10/394,086, filed Mar. 21, 2003, which claims priority under 35 U.S.C. 120 of international application no. PCT/DK01/00634 filed Oct. 2, 2001 and claims priority under 35 U.S.C. 119 of Danish application no. PA 2000 01456 filed on Oct. 2, 2000; Danish application no. PA 2001 00262 filed on Feb. 16, 2001; Danish application no. PA 2001 00430 filed on Mar. 14, 2001; Danish application no. PA 2001 00751 filed on May 14, 2001; U.S. application No. 60/238,944 filed on Oct. 10, 2000; U.S. provisional application No. 60/271,581 filed on Feb. 26, 2001 and U.S. provisional application No. 60/276,322 filed on Mar. 16, 2001, the contents of which are fully incorporated herein by reference.

### FIELD OF THE INVENTION

The present invention relates to methods for cultivating mammalian cells and for producing recombinant proteins in large- or industrial-scale cultures of such cells.

### BACKGROUND OF THE INVENTION

The proteins involved in the clotting cascade, including, e.g., Factor VII, Factor VIII, Factor IX, Factor X, and Protein C, are proving to be useful therapeutic agents to treat a variety of pathological conditions. Because of the many disadvantages of using human plasma as a source of pharmaceutical products, it is preferred to produce these proteins in recombinant systems. The clotting proteins, however, are subject to a variety of co- and post-translational modifications, including, e.g., asparagine-linked (N-linked) glycosylation; O-linked glycosylation; and γ-carboxylation of glu residues. For this reason, it is preferable to produce them in mammalian cells, which are able to modify the recombinant proteins appropriately. Mammalian cell culture, however, has traditionally been performed in the presence of animal serum or animal-derived components such as albumin, transferrin etc. Methods for serum-free cultivation have produced variable results. In particular, cultivation of cells in the absence of serum from initiation of the culture until attainment of large-scale production volumes has been problematic.

Thus, there is a need in the art for methods for large-scale mammalian cell culture free of serum or other animal-derived components to produce industrial quantities of clotting proteins, particularly recombinant human Factor VII or Factor VII-related polypeptides. There is also a need in the art for methods for industrial-scale mammalian culture free of animal derived components or ingredients wherein the yield of protein is maintained or increased compared to small scale or laboratory scale amounts of expressed protein, or wherein the yield of protein is increased compared to amounts of expressed protein when produced in culture containing animal-derived components or ingredients, in particular serum.

### SUMMARY OF THE INVENTION

The present invention provides methods for large-scale production of Factor VII or a Factor VII-related polypeptide in mammalian cells, which are carried out by the steps of:

(i) inoculating Factor VII-expressing mammalian cells into a culture vessel containing medium lacking animal-derived components and propagating said culture at least until the cells reach a predetermined density;

(ii) transferring said propagated seed culture to a large-scale culture vessel containing medium lacking animal-derived components;

(iii) propagating said large-scale culture in medium lacking animal-derived components, at least until said cells reach a predetermined density;

(iv) maintaining the culture obtained in step (iii) in medium lacking animal-derived components, under conditions appropriate for Factor VII expression; and

(v) recovering the Factor VII from the maintained culture.

In some embodiments, the invention relates to cultivation of suspension-competent mammalian cells in medium lacking animal-derived components. In some embodiments, the cells have been adapted to grow in medium lacking animal-derived proteins and/or in suspension culture. In some embodiments, the cells used have been adapted to grow in suspension culture in medium lacking animal-derived components prior to inoculation in step (i). In another aspect, the present invention is based on the discovery that the use of macroporous carriers having a positive surface charge provides a suitable environment for the propagation of suspension-competent cells in the absence of animal-derived components and allows high-level production of desired proteins by such cells.

In some embodiments, the method further comprises, prior to step (ii), that step (i) is repeated using seed culture vessels of progressively increasing size.

The present invention also provides methods for large-scale production of a Factor VII or a Factor VII-related polypeptide in mammalian cells, which are carried out by the steps of:

(i) Inoculating cells into a seed culture vessel containing medium lacking animal-derived components and propagating said seed culture at least until the cells reach a minimum cross-seeding density;

(ii) Transferring said propagated seed culture to a large-scale culture vessel containing medium lacking animal-derived components; and

(iii) Propagating said large-scale culture in medium lacking animal-derived proteins, at least until said cells reach a predetermined density.

In some embodiments, the method further comprises:

(iv) Maintaining the culture obtained in step (iii) in medium lacking animal-derived components by regular harvesting of the culture medium and replacement by fresh medium.

In some embodiments, the method is a standard microcarrier process and further comprises:

(iv) maintaining the culture obtained in step (iii) in medium lacking animal-derived components by regular harvesting of part of the culture supernatant after sedimentation of the cell-containing carriers and replacement by fresh medium.

In some embodiments, the method is a standard microcarrier process and further comprises:

(v) cooling the culture to a pre-determined temperature (from 5 to 30° C., such as, e.g., from 5 to 20° C., or from 5 to 15° C. or to about 10° C.) below the temperature setpoint of the cultivation) before the sedimentation of carriers.

The present invention also provides methods for large-scale production of Factor VII or a Factor VII-related polypeptide in mammalian cells, which are carried out by the steps of:

(i) providing a mammalian cell expressing Factor VII or a Factor VII-related polypeptide;

(ii) inoculating said cell into a seed culture vessel containing medium lacking animal-derived components and propagating said seed culture at least until the cells reach a minimum cross-seeding density;

(iii) transferring said propagated seed culture to a large-scale culture vessel containing medium lacking animal-derived components; and

(iv) maintaining said large-scale culture in medium lacking animal-derived components, at least until said cells reach a minimum desired density, under conditions in which said Factor VII or said Factor VII-related polypeptide is produced by said culture.

In some embodiments, the cells have been adapted to grow in suspension culture in medium lacking animal-derived components prior to inoculation in step (i). Preferably, a Factor VII or a Factor VII-related polypeptide is produced at a level at least about 1 mg/l of culture, such as, e.g., at least about 2.5 mg/l of culture, or at least about 5 mg/l of culture, or at least about 8 mg/l of culture.

In some embodiments, the cells produce a desired polypeptide, preferably a clotting factor and most preferably human Factor VII or a human Factor VII-related polypeptide, including, without limitation, wild-type Factor VII, S52A-Factor VII, S60A-Factor VII, R152E-Factor VII, S344A-Factor VII, and Factor VIIa lacking the Gla domain.

In some embodiments, the process of the present invention is a micro carrier-type process; in other embodiments, the method is a suspension cell-type process.

In some embodiments, the microcarrier process is a standard microcarrier process. In some embodiments of the standard microcarrier process, part of the culture supernatant is harvested with regular intervals after sedimentation of the cell-containing carriers and replaced with fresh medium. In some embodiments, the standard microcarrier process further comprises cooling of the culture to a temperature (e.g. from 5° C. to 30° C., or from 5° C. to 20° C., or from 5° C. to 15° C., or to about 10° C.) below the temperature setpoint of the cultivation immediately before each sedimentation of carriers. The cooling step is done within 10-240 minutes, such as, e.g, 20-180 minutes, or 30-120 minutes, before sedimenting the cell-containing microcarriers. In some embodiments, the method is a microcarrier perfusion process. In some embodiments, the method is a microcarrier process and the microcarrier is a macroporous carrier.

In some embodiments, the method is carried out by the steps of:

(i) inoculating Factor VII-expressing or Factor VII-related polypeptide-expressing mammalian cells into a culture vessel containing medium lacking animal-derived components and propagating said culture at least until the cells reach a predetermined density;

(ii) transferring said propagated seed culture to a large-scale culture vessel containing (a) medium lacking animal-derived components and (b) macroporous carriers, under conditions in which said cells migrate into the carriers;

(iii) propagating said large-scale culture in medium lacking animal-derived components, at least until said cells reach a predetermined density;

(iv) maintaining the culture obtained in step (iii) in medium lacking animal-derived components, under conditions appropriate for Factor VII expression; and

(v) recovering the Factor VII from the maintained culture.

Preferably, the microcarriers:

(a) have an overall particle diameter between about 150 and 350 um; and

(b) have a positive charge density of between about 0.8 and 2.0 meq/g.

Preferably, the macroporous carriers:

(a) have an overall particle diameter between about 150 and 350 um;

(b) have pores having an average pore opening diameter of between about 15 and about 40 um; and

(c) have a positive charge density of between about 0.8 and 2.0 meq/g.

In some embodiments, the microcarriers are dextran-based; in some embodiments, the macroporous carriers are cellulose-based; in some embodiments, the carriers comprise surface DEAE groups that impart said charge density.

In some embodiments, the suspension cell process is a perfusion process; in other embodiments, the method is a batch/draw-fill process.

In some embodiments, the batch/draw-fill process is a simple batch process; in other embodiments, the method is a fed-batch process; in yet other embodiments, the method is a draw-fill process.

In some embodiments, the cells used are BHK cells; in other embodiments, the cells are CHO cells; in other embodiments, the cells are HEK cells; in other embodiments, the cells are COS cells; in other embodiments, the cells are HeLa cells. Preferred are BHK and CHO cells.

In some embodiments, the CHO cells are grown to a selected density at a first temperature. When the selected cell density has been reached, the temperature is lowered to a second temperature. In some embodiments, the first temperature is from about 30-37° C. and the second temperature is from about 30-36° C.; preferably, the first temperature is about 37° C. for CHO cells and about 36° C. for BHK cells, and the second temperature is about 32° C. for both CHO and BHK cells.

In some embodiments, sodium butyrate is added at a specified concentration at a specific cell concentration in the culture vessel.

In some embodiments of a fed-batch or a fed-batch draw-fill process, the feed to be used is a concentrated solution of glucose; in other embodiments, the feed is a concentrated feed consisting of the cell medium at a×10-50 concentration. In some embodiments, the feed is modified to ameliorate that some of the media components may be detrimental to the cells or simply will not dissolve at a high concentration. In some embodiments, the feed is added as a single pulse (once, twice, three times, etc., a day); in other embodiments, the feed is fed gradually throughout a 24-hour period. In some embodiments, the culture vessel contains a glucose sensor that will control the feed rate to maintain a constant glucose concentration in the vessel; in other embodiments, the culture vessel contains a one-line biomass monitor (Aber Instrument) (See, for example: Case studies on the use of on-line and off-line radio-frequency impedance methods in cell culture. Claire L. Harding, John P. Carvell and Yue Guan. Presented at the 16^{th} ESACT Meeting, Lugano, 25 to 29 Apr. 1999).

In some embodiments, the cells used in practicing the present invention are adapted to suspension growth in medium lacking animal-derived components, such as, e.g., a medium lacking serum, or a medium lacking animal-derived components and proteins. In a particularly preferred embodiment, the host cells are BHK 21 or CHO cells that have been engineered to express human Factor VII or human Factor VII-related polypeptides and that have been adapted to grow in the absence of serum or animal-derived components.

In some embodiments, the protein expressed is human Factor VII. In other embodiments, the protein expressed is Factor VII having substantially the same or improved biological activity compared to wild-type FVII. In other embodiments, the protein expressed is a Factor VII-related polypeptide having modified or reduced biological activity compared to wild-type FVII.

### LIST OF FIGURES

FIG. 1 shows a diagram of a standard microcarrier process

FIG. 2 shows a diagram of a microcarrier perfusion process.

FIG. 3 shows a diagram of a simple draw-fill process.

FIG. 4 shows a diagram of the different types of processes suitable according to the present invention.

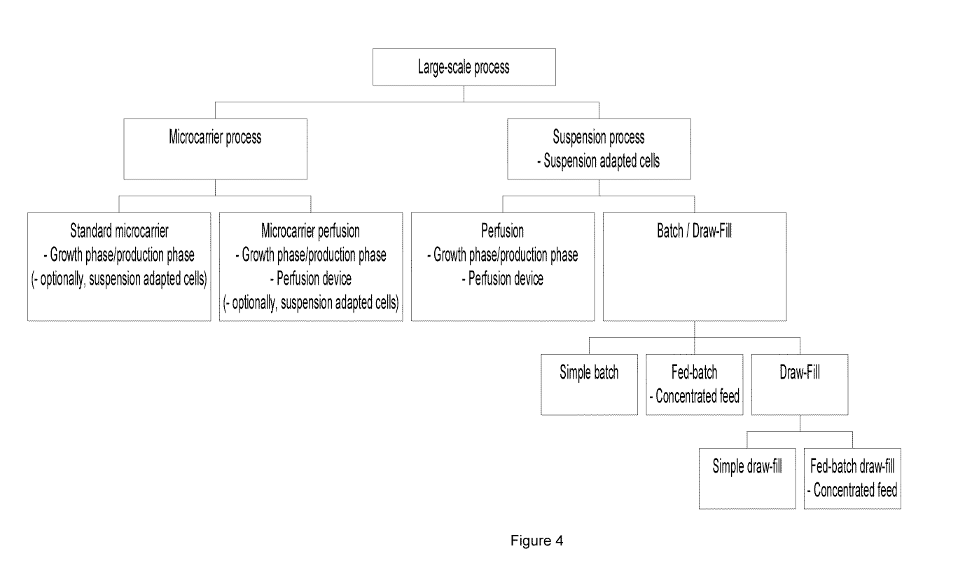

### DETAILED DESCRIPTION OF THE INVENTION

The present invention provides methods for large-scale cultivation of mammalian cells, particularly to produce industrial amounts of desired polypeptides that are expressed by such cells. The methods are carried out by the steps of:

(i) inoculating cells into a seed culture vessel containing culture medium lacking animal-derived components and propagating the seed culture at least until the cells reach a minimum cross-seeding density;

(ii) transferring the propagated seed culture to a large-scale culture vessel containing culture medium lacking animal-derived components; and

(iii) propagating the large-scale culture in medium lacking animal-derived components, at least until said cells reach a useful density.

In some embodiments, the methods further comprise the step of:

(iv) maintaining the culture obtained in step (iii) in medium lacking animal-derived components by regular harvesting of the culture medium and replacement by fresh medium.

The below-described processes are applicable for any cell type in any formulation of medium lacking animal-derived components The first two processes described are for cells attached to and/or immobilised in a macroporous carrier.

### Microcarrier Processes:

Two types of microcarrier processes may be used. These are:

1. Standard Microcarrier Process

2. Microcarrier Perfusion Process.

### Standard Microcarrier Process:

This process is operated in two distinct phases.

1. Growth Phase.

2. Production Phase.

### Growth Phase

In a standard microcarrier-process the cells are inoculated into a seed culture vessel containing culture medium lacking animal-derived components and propagated until the cells reach a minimum cross-seeding density. Subsequently, the propagated seed culture is transferred to a large-scale culture vessel containing (a) culture medium lacking animal-derived components and (b) microcarriers, under conditions in which the carriers are fully colonised by the cells, for example by migrating into the carriers in case of a process using macroporous carriers.;

In this growth phase, the cells are grown on microcarriers until the carriers are fully colonised. The medium exchange is performed by allowing the microcarriers to settle to the bottom of the culture vessel, after which a predetermined percentage of the tank volume is removed and a corresponding percentage tank volume of fresh medium is added to the vessel. The microcarriers are then re-suspended in the medium and this process of medium removal and replacement are repeated at a predetermined interval, for example every 24 hours. The amount of replaced medium depends on the cell density and may typically be from 10-95%, preferably from 25% to 80%, of the tank volume as shown in Table 1 below.

When the cell density reaches the value suitable for protein expression, 60-95% of the tank medium in the tank is changed every 24 hours, preferably 80%. A 80% medium exchange is also preferably used in the production phase.

An outline of this aspect of the process is shown in Table 1.

TABLE 1

| Setpoint | Range | Preferred range | More preferred Value |
|---|---|---|---|
| PH | 6-8 | 6.6-7.6 | 7.0 |
| Temperature | 28-40° C. | 34-38° C. | 36-37° C. |
| Dissolved Oxygen | 10-90% of saturation | 20-80% of saturation | 50% of saturation |
| Tension | | | |
| Daily Medium | | | |
| Change: | | | |
| % of medium | 10-35% of medium | 25% of medium | 25% of medium exchanged at |
| changed at | exchanged | exchanged at 0.4-1.0 × | 0.5 × 10⁶ cells ml−1 |
| | at 0.4-1.0 × 10⁶ | 10⁶ cells ml−1 | |
| | cells ml−1 | | |
| % of medium | 30-70% of medium | 50% of medium exchanged | 50% of medium exchanged at |
| changed at | exchanged | changed at 0.7-3.0 × | 1.0 × 10⁶ cells ml−1 |
| | at 0.7-3.0 × 10⁶ | 10⁶ cells ml−1 | |
| | cells ml−1 | | |
| % of medium | 60-90% of medium | 80% of medium exchanged | 80% of medium exchanged at |
| changed at | exchanged | changed at 1.0-12.0 × | 2.0-10 × 10⁶ cells ml−1 |
| | at 1.0-12.0 × 10⁶ | 10⁶ cells ml−1 | |
| | cells ml−1 | | |

Some of the setpoints that are suitable for production of FVII are not necessarily suitable for the initial growth of the cells, either in seed culture or on the microcarriers. For example, temperature, DOT, and/or pH may be different for the two phases. The medium exchange at this stage, even if at the same level as in the production phase, is done to keep the cells alive and growing.

### Production Phase

In the growth phase the culture is propagated until the cells reach a density of 1-12×10⁶ cells per ml. Reaching this density, the culture enters the production phase. Setpoints may also be changed at this point and set at values suitable for production of FVII.

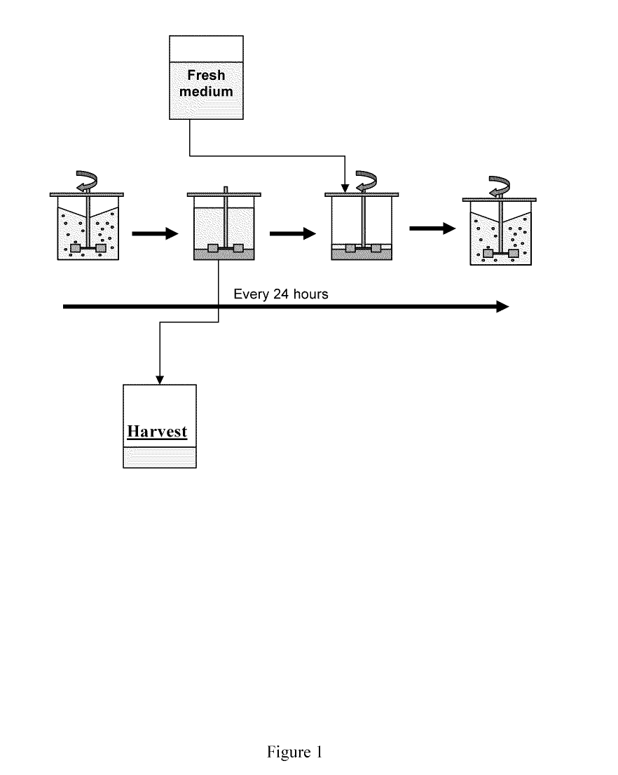

A diagram of the process is shown in FIG. 1.

The medium exchange is performed by allowing the microcarriers to settle to the bottom of the tank, after which the selected % of the tank volume is removed and a corresponding % tank volume of fresh medium is added to the vessel. From 25-90% of the tank volume may be exchanged; preferably, 80% of the tank volume is exchanged. The microcarriers are then re-suspended in the medium and this process of medium removal and replacement are repeated every 10-48 hours; preferably, every 24 hours.

An outline of this aspect of the process is shown in Table 2.

TABLE 2

| Setpoint | | Preferred range | More preferred Value |
|---|---|---|---|
| PH | 6-8 | 6.6-7.6 | 7.0 for CHO and 6.7-6.9 |
| | | | for BHK |
| Temperature | 26-40° C. | 30-37° C. | 32° C. for CHO and |
| | | | 36° C. for BHK |
| Dissolved Oxygen Tension | 10-90% of saturation | 20-80% of saturation | 50% |
| % of medium changed | 25-90% of medium | 80% of medium | 80% of medium |
| | exchanged every | changed every 10-48 | changed every 24 |
| | 10-48 hours | hours | hours |

### Optional (1)

A drop in temperature set point of the cultivation may be employed when entering—and during—the production phase. Temperature, operating pH and medium exchange frequency are optimised. In particular, a drop in temperature is preferred when using a CHO cell line. Temperature ranges and preferred values in growth and production phase, respectively, can be seen from Tables 1 and 2. A temperature of about 32° C. would be preferred for a CHO cell line during the production phase.

### Optional (2)

A cooling step may be applied immediately before each sedimentation of carriers. The culture is cooled to a predetermined temperature below the temperature setpoint of the cultivation (e.g. from 5° C. to 30° C., or from 5° C. to 20° C., or from 5° C. to 15° C., or to about 10° C. below setpoint). The cooling step is done within 10-240 minutes, such as, e.g, 20-180 minutes, or 30-120 minutes, before sedimenting the cell-containing microcarriers.

The step is typically carried out as follows: The bioreactor is cooled and the temperature is monitored. When the bioreactor reaches a pre-determined temperature below the setpoint temperature, such as, e.g., 10° C. below the set point of the culturing, the stirring of the bioreactor contents is stopped and the cell-containing carriers are sedimented. When media exchange has taken place, the temperature is again regulated to the setpoint of the culturing. The fresh media being added is typically pre-warmed to a temperature close to the setpoint of the cultivation.

### Microcarrier Perfusion Process:

This process resembles the standard microcarrier process and is again operated in two distinct phases.

1. Growth Phase.

2. Production Phase.

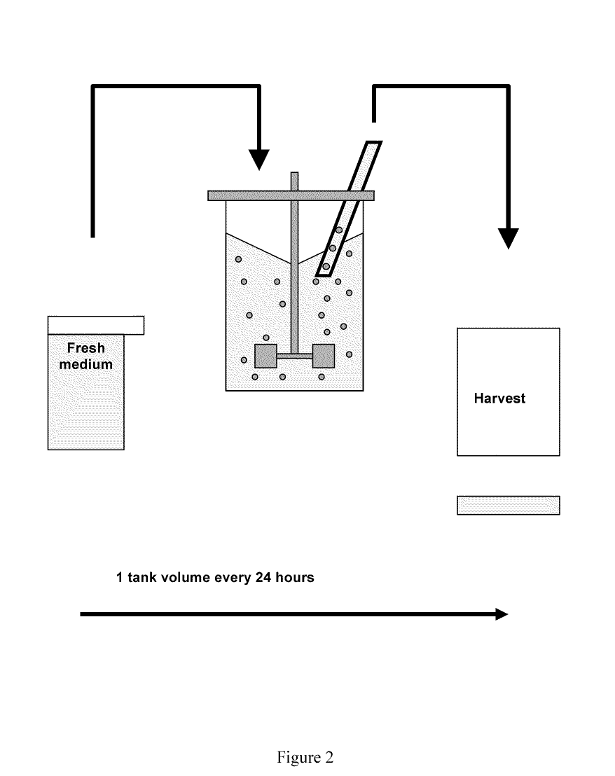

The main difference between this and the standard process described above is the method employed to change the culture medium. In the previously described standard microcarrier process a defined percentage of the tank volume, for example 80% of the total tank volume, is changed all at once. In a perfusion process the medium is added continuously and an equal volume of harvest is also removed continuously. Essentially, the medium (defined % tank volume) is changed gradually over a predetermined period of time, for example a 24-hour period. This is shown in the diagram in FIG. 2.

The microcarriers are kept in the vessel by using a separation device (or perfusion device) that allows the medium to leave the vessel but retains the microcarriers within the tank.

### Growth Phase

As described for standard microcarrier process except for the gradual medium exchange. The exchange of medium is given as % tank volume per day, i.e., 24 hours.

An outline of this aspect of the process is shown in Table 3.

TABLE 3

| Setpoint | Range | Preferred range | More preferred Value |
|---|---|---|---|
| PH | 6-8 | 6.6-7.6 | 7.0 |
| Temperature | 28-40° C. | 34-38° C. | 36-37° C. |
| Dissolved Oxygen | 10-90% of saturation | 20-80% of saturation | 50% |
| Tension | | | |
| Medium Flow Rate | | | |
| % tank volume per | 10-35% of medium | 25% of medium perfused | 25% of medium perfused |
| day (24 hours) at | perfused at 0.4-1.0 × | at 0.4-1.0 × 10⁶ | at 0.5 × 10⁶ cells |
| | 10⁶ cells ml−1 | cells ml−1 | ml−1 |
| % tank volume per | 30-70% of medium | 50% of medium perfused | 50% of medium perfused |
| day (24 hours) at | perfused at 0.7-3.0 × | at 0.7-3.0 × 10⁶ | at 1.0 × 10⁶ cells |
| | 10⁶ cells ml−1 | cells ml−1 | ml−1 |
| % tank volume per | 60-95% of medium | 80% of medium perfused | 80% of medium perfused |
| day (24 hours) at | perfused at 1.0-12.0 × | at 1.0-12.0 × 10⁶ | at 2.0-10 × 10⁶ |
| | 10⁶ cells ml−1 | cells ml−1 | cells ml−1 |

Again, even though we are perfusing the culture at high medium exchange (e.g., 80% tank volume) at an early stage this is not considered to be the production phase. This is because some of the setpoints that are suitable for production of FVII may not be suitable for the initial growth of the cells on the microcarriers. The perfusion with fresh medium at this stage is done to keep the cells alive and growing. For the purposes of comparison with the ‘standard microcarrier process’ the flow rate of medium is expressed in terms of percentage tank volume of medium per day (24 hours).

### Production Phase

As in the above-described process, in the growth phase the culture is propagated until the cells reach a density of 1-12×10⁶ cells per ml. Reaching this density, the culture enters the production phase. Setpoints may also be changed at this point and set at values suitable for production of FVII.

A diagram of the process is shown in FIG. 2.

The medium perfusion is performed continuously. For the purposes of comparison with the ‘standard microcarrier process’ the flow rate of medium is expressed in terms of percentage tank volume of medium per defined period of time. Medium perfusion may be from 25-90% tank volume per 10-48 hours; preferably, the medium perfusion is 80% per 10-48 hours, more preferred 80% tank volume every 24 hours.

An outline of this aspect of the process is shown in Table 4.

TABLE 4

| Setpoint | Range | Preferred range | More preferred Value |
|---|---|---|---|
| PH | 6-8 | 6.6-7.6 | 7.0 for CHO and 6.7-6.9 |
| | | | for BHK |
| Temperature | 26-40° C. | 30-37° C. | 32° C. for CHO and |
| | | | 36° C. for BHK |
| Dissolved Oxygen | 10-90% | 20-80% | 50% |
| Tension | | | |
| % tank volume of | 25-90% of medium | 80% of medium perfused | 80% of medium perfused |
| medium perfused | perfused every 10-48 | every 10-48 | every 24 hours |
| | hours | hours | |

### Perfusion Devices

Suitable means for achieving retention of carriers is a settling device inside the vessel, e.g. a dip tube.

### Optional

A drop in temperature may be employed when entering—and during—the production phase. Temperature, operating pH and medium exchange frequency are optimised. In particular when using a CHO cell line, a drop in temperature is preferred. Temperature ranges and preferred values in growth and production phase, respectively, can be seen from Tables 3 and 4. A temperature of about 32° C. would be preferred for a CHO cell line during the production phase.

### Suspension Cell Processes:

There are two main options for a suspension cell process which are:—

1. Perfusion Process.

2. Batch/Draw-Fill Process.

### Perfusion Process:

This process resembles the process outlined for microcarrier perfusion. The main difference is 1) that the cells are grown freely suspended without being immobilised in carriers and 2) the nature of the perfusion device employed to retain the cell in the culture vessel. The process is again operated in two distinct phases.

1. Growth Phase.

2. Production Phase.

### Growth Phase

In a suspension cell-perfusion process the cells are inoculated into a seed culture vessel containing culture medium lacking animal-derived components and propagated until the cells reach a minimum cross-seeding density. Subsequently, the propagated seed culture is transferred to a large-scale culture vessel containing culture medium lacking animal-derived components and propagated until at least a predetermined cell density is reached.

In this phase the cells are grown in suspension to allow the cell number within the culture vessel to increase to a predetermined or critical value. The medium exchange is performed by continuously perfusing the culture vessel with fresh medium.

The amount of perfused medium depends on the cell density and may typically be from 10-95%, preferably from 25% to 80%, of the tank volume per day (24 hours) as shown in Table 5 below.

When the cell density reaches the value suitable for initiation of production phase, 60-95% of the tank medium in the tank is changed every 24 hours, preferably 80%. An 80% medium exchange is also preferably used in the production phase.

Again, even though we are perfusing the culture at an early stage this is not considered to be the production phase. This is because some of the setpoints that are suitable for production of FVII are not suitable for the initial growth of the cells on the macroporous carriers. The perfusion with fresh medium at this stage is done to keep the cells alive and growing.

An outline of this aspect of the process is shown in Table 5.

TABLE 5

| Setpoint | Range | Preferred range | More preferred Value |
|---|---|---|---|
| PH | 6-8 | 6.6-7.6 | 7. |
| Temperature | 28-40° C. | 34-38° C. | 36-37° C. |
| Dissolved Oxygen | 10-90% | 20-80% | 50% |
| Tension | | | |
| Medium Flow Rate | | | |
| % tank volume per | 10-35% volume at | 25% of tank volume | 25% of tank volume |
| day at | 0.4-1.0 × 10⁶ cells ml−1 | perfused at 0.4-1.0 × | perfused at 0.5 × 10⁶ |
| | | 10⁶ cells ml−1 | cells ml−1 |
| % tank volume per | 30-70% volume at | 50% of tank volume | 50% of tank volume |
| day at | 0.7-3.0 × 10⁶ cells ml−1 | perfused at 0.7-3.0 × | perfused at 1.0 × 10⁶ |
| | | 10⁶ cells ml−1 | cells ml−1 |
| % tank volume per | 60-95% volume at | 80% of tank volume | 80% of tank volume |
| day at | 1.0-12.0 × 10⁶ cells ml−1 | perfused at 1.0-12.0 × | perfused at 2.0-10 × |
| | | 10⁶ cells ml−1 | 10⁶ cells ml−1 |

### Production Phase

In the growth phase the culture is propagated until the cells reach a density of 1-12×10⁶ cells per ml. Reaching this density, the culture enters the production phase. Setpoints may also be changed at this point and set at values suitable for production of FVII.

The medium perfusion is performed continuously. For the purposes of comparison the flow rate of medium is expressed in terms of percentage tank volume of medium per defined period of time. (A more standard unit would be litres per day). Medium perfusion may be from 10-200% tank volume per 10-48 hours; preferably, the medium perfusion is 80% per 10-48 hours, more preferred 80% tank volume every 24 hours.

An outline of this aspect of the process is shown in Table 6.

TABLE 6

| Setpoint | Range | Preferred range | More preferred Value |
|---|---|---|---|
| PH | 6-8 | 6.6-7.6 | 7.0 for CHO and 6.7-6.9 |
| | | | for BHK |
| Temperature | 26-40° C. | 30-37° C. | 32° C. for CHO and |
| | | | 36° C. for BHK |
| Dissolved Oxygen | 10-90% | 20-80% | 50% |
| Tension | | | |
| % tank volume of | 10-200% of tank volume | 80% of tank volume | 80% of tank volume |
| medium perfused | perfused in 10-48 | perfused in 10-48 | perfused every 24 |
| | hours | hours | hours |

### Perfusion Devices

Cell retention within the culture vessel may be achieved using a number of cell retention devices. The following sets of apparatus may all be used for this process.

1. External settling head.

2. Internal settling head

3. Continuous centrifuge

4. Internal or external spin filter.

5. External filter or hollow fibre cartridge.

6. Ultrasonic cell separating device

7. A length of pipe inside the culture vessel.

### Optional

A drop in temperature may be employed when entering—and during—the production phase. Temperature, operating pH and medium exchange frequency are optimised. In particular when using a CHO cell line, a drop in temperature is preferred. Temperature ranges and preferred values in growth and production phase, respectively, can be seen from Tables 5 and 6. A temperature of about 32° C. would be preferred for a CHO cell line during the production phase.

### Batch/Draw Fill Process:

These are probably the simplest type of fermentations to operate and there are three main options for a suspension cell process using this format:

1. Simple Batch Process

2. Fed-Batch Process

3. Draw-Fill Process

### Simple Batch Process:

In a simple batch process the cells are inoculated into a seed culture vessel containing culture medium lacking animal-derived components and propagated until the cells reach a minimum cross-seeding density. Subsequently, the propagated seed culture is transferred to a large-scale culture vessel containing culture medium lacking animal-derived components. The culture vessel is then operated until the nutrients in the medium are exhausted.

An outline of this aspect of the process is shown in Table 7.

TABLE 7

| Setpoint | Range | Preferred range | More preferred Value |
|---|---|---|---|
| PH | 6-8 | 6.6-7.6 | 7.0 |
| Temperature | 28-40° C. | 30-37° C. | 36-37° C. |
| Dissolved Oxygen | 10-90% | 20-80% | 50% |
| Tension | | | |
| Temperature drop to | 26-39° C. | 30-36° C. | 32° C. |
| (Optional) | | | |
| Temperature drop at | 0.5-12.0 × 10⁶ cells | 0.5-12.0 × 10⁶ cells | 2.0-10 × 10⁶ cells ml⁻¹ |
| | ml⁻¹ | ml⁻¹ | |

### Optional

An optional aspect of the process is the use of a reduced operating temperature. Such a batch process would consist of an initial growth phase at a specific temperature suitable for growth of the used cell line followed by a drop in operating temperature at a predetermined cell density, for example 1-12×10⁶ cells ml⁻¹. This is particularly relevant for the CHO cell lines. A preferred batch process for CHO would consist of an initial growth phase at 37° C. followed by a drop in operating temperature at 1-12×10⁶ cells ml⁻¹, preferably 2-10×10⁶ cells ml⁻¹. Preferably, the temperature drop would be from 37° C. to 32° C. in case of CHO cells.

The time of harvest has to be determined. A traditional batch is operated until all nutrients become exhausted. However, this typically causes cell lysis, which either can be damaging to the product or may cause problems to purification.

### Fed-Batch Process:

As stated previously a simple batch process consists inoculating a culture vessel with cells and operating the tank until the nutrients in the medium are exhausted. A batch process such as this can be extended by feeding a concentrated solution of nutrients to the tank. This extends the process time and ultimately leads to an increase in FVII production within the culture vessel.

The most critical nutrient in the culture vessel is the glucose concentration. The control and initiation of the feed is linked to the level of this nutrient. When the glucose concentration falls below a critical value a feed is initiated and the amount of feed added is sufficient to raise the glucose concentration back to this critical value.

An outline for a Fed-batch aspect process is shown in Table 8.

TABLE 8

| Setpoint | Range | Preferred range | More preferred Value |
|---|---|---|---|
| pH | 6-8 | 6.6-7.6 | 7.0 |
| Temperature | 28-40° C. | 30-37° C. | 36-37° C. |
| Dissolved Oxygen | 10-90% | 20-80% | 50% |
| Tension | | | |
| Temperature drop to | 26-39° C. | 30-36° C. | 32° C. |
| (Optional) | | | |
| Temperature drop at | 0.5-12.0 × 10⁶ cells | 0.5-12.0 × 10⁶ cells | 2.0-10 × 10⁶ cells ml⁻¹ |
| | ml⁻¹ | ml⁻¹ | |
| Feed initiated at glucose | 6-0 gl⁻¹ | 3-0 gl⁻¹ | When glucose <2 gl⁻¹ |
| concentration | | | |

An optional aspect of the process is the use of a reduced operating temperature. Such a fed batch process would consist of an initial growth phase at a specific temperature suitable for growth of the used cell line followed by a drop in operating temperature at a predetermined cell density, for example 1-12×10⁶ cells ml⁻¹. This is particular relevant for the CHO cell lines. A preferred fed batch process for CHO would consist of an initial growth phase at 37° C. followed by a drop in operating temperature at 1-12×10⁶ cells ml⁻¹, preferably 2-10×10⁶ cells ml⁻¹. Preferably, the temperature drop would be from 37° C. to 32° C. in case of CHO cells.

Like in a simple batch process the time of harvest has to be determined as a balance between the longest possible operation of the tank and the risk of cell lysis.

### Feed Composition & Addition Strategy

The simplest feed suitable for use would be a concentrated solution of glucose. However, glucose feeding alone will only extend the batch phase for a short length of time. This is because another nutrient such as an amino acid or lipid or vitamin will then become exhausted. For this reason a concentrated feed would be preferable. The simplest concentrated feed suitable for use would be the cell medium at a×10-50 concentration.

An outline of this aspect is shown in Table 9.

TABLE 9

| | | | More preferred |
|---|---|---|---|
| Feed Compositions | Range | Preferred range | Value |
| Glucose | 50-1000 gl⁻¹ | 50-500 gl⁻¹ | 200 gl⁻¹ |
| Medium Concentrate | X 10-50 | X 2-20 | X 10 |
| Modified concentrate | 0-x50 | 0-x 20 | 0-x10 |
| so individual medium | | | |
| components are in the | | | |
| ranges of:- | | | |
| Most Probable | | | |
| Composition of a | | | |
| Concentrate | | | |
| Buffer | 0-x50 | 0-x20 | x1 |
| Insulin | 0-x50 | 0-x20 | x1 |
| Lipids | 0-x50 | 0-x20 | x1 |
| Iron source | 0-x50 | 0-x20 | x1 |
| Cysteine and | 0-x50 | 0-x20 | x1 |
| Cystine | | | |
| Plant hydrolysates | 0-x50 | 0-x20 | x1 |
| All other components | 0-x50 | 0-x20 | x10 |

Unfortunately, some of the medium components may be detrimental to the cell or simply will not dissolve at a high concentration. For this reason the feed might need modification to keep these problem components at a low level.

The method of addition of the feed is also a variable. The feed can be added either as a single pulse (once, twice, three times etc., a day) or can be fed gradually throughout a 24-hour period. An advanced feed option would be to have some form of glucose sensor in the culture vessel that will control the feed rate to maintain a constant glucose concentration in the vessel.

The time of harvest has to be determined. A traditional, or simple, batch is operated until all nutrients become exhausted. This is not generally a problem in a Fed-Batch system. However, the process cannot be sustained indefinitely due to the accumulation of toxic metabolites. This leads to a decrease in cell viability and ultimately cell lysis. This may cause damage to the product or cause problems to subsequent purification.

### Draw-Fill Process:

Two types of Draw-Fill will be described here. These are:

1. Simple Draw-Fill

2. Fed Batch Draw-Fill

Simple Draw-Fill:

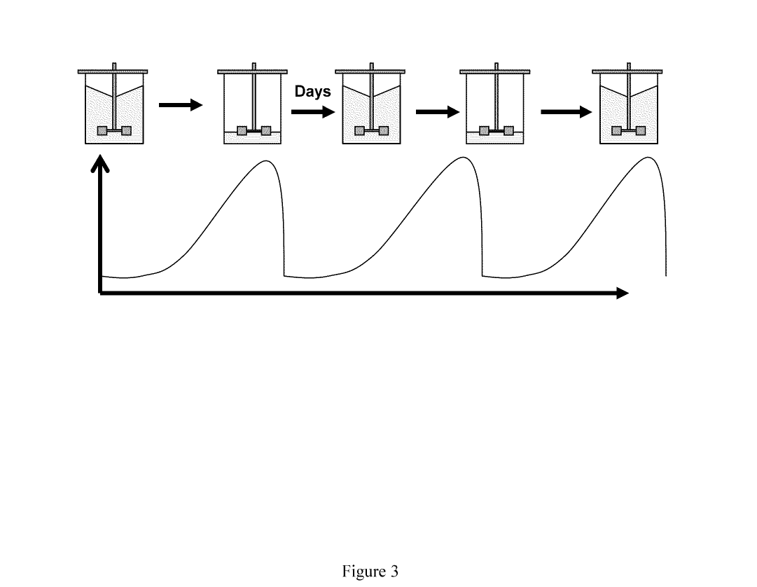

This process closely resembles a repeated batch fermentation (see FIG. 3). In batch fermentation the cells grow in the culture vessel and the medium is harvested at the end of the run. In a Draw-Fill process the culture vessel is harvested before any of the nutrients become exhausted. Instead of removing all of the contents from the vessel, only a proportion of the tank volume is removed (typically 80% of the tank volume). After the harvest, the same volume of fresh medium is added back to the vessel. The cells are then allowed to grow in the vessel once more and another 80% harvest is taken a set number of days later. In epeated batch processes the cells left in the vessel after a harvest may be used as the inoculum for the next batch.

An outline for a Draw-Fill process is shown in Table 10. The process is operated in two phases. The first phase of the process is operated identically to a simple batch process. After the first harvest, the culture vessel is again operated as a simple batch process; however, the length of the batch is shorter than the first batch because of the higher initial cell density. Theses short ‘repeated batch phases’ are continued indefinitely.

A simple outline of a draw-fill to be employed is:

Initial Batch Phase
i. Inoculate vessel and allow cells to grow at a temperature suitable for growth.
ii. Drop temperature to a temperature suitable for expression at a predetermined cell density.
iii. 7 days after inoculation remove a predetermined, e.g. 80%, of the tank volume and replace with the same volume of fresh medium.

Repeated Batch Phase
iv. Increase temperature to a temperature suitable for growth and allow the cells to grow.
v. Drop temperature to a temperature suitable for expression at a predetermined cell density.
vi. 5 days after the start of this phase remove a predetermined, e.g. 80%, of the tank volume and replace with the same volume of fresh medium.
vii. Go to step iv.

In a preferred embodiment, the cell line is a CHO cell line. A simple outline of a draw-fill we might employ for a CHO cell line is:

Initial Batch Phase
viii. Inoculate vessel and allow cells to grow at 37° C.
ix. Drop temperature to 32° C. at 2-10×10⁶ cells ml⁻¹.
x. 7 days after inoculation remove 80% of the tank volume and replace with the same volume of fresh medium.

Repeated Batch Phase
xi. Increase temperature to 37° C. and allow the cells to grow.
xii. Drop temperature to 32° C. at 2-10×10⁶ cells ml⁻¹.
xiii. 5 days after the start of this phase remove 80% of the tank volume and replace with the same volume of fresh medium.
xiv. Go to step xi.

The culture vessel may be operated within a broad range of cycle times and a broad range of draw-fill volumes.

Ranges and preferred values can be seen from Table 10.

TABLE 10

| Setpoint | Range | Preferred range | More preferred Value |
|---|---|---|---|
| Initial Batch Phase | | | |
| PH | 6-8 | 6.6-7.6 | 7.0 for CHO and 6.6-7.4 |
| | | | for BHK |
| Temperature | 28-40° C. | 30-37° C. | 37° C. for CHO and |
| | | | 36° C. for BHK |
| Temperature drop | | | |
| (OPTIONAL) | | | |
| Temperature | 26-39° C. | 30-36° C. | 32° C. |
| drop to | | | |
| Temperature | 0.5-12.0 × 10⁶ cells | 0.5-12.0 × 10⁻⁶ cells | 2.0-10 × 10⁻⁶ cells ml⁻¹ |
| drop at | ml⁻¹ | ml⁻¹ | |
| DOT | 10-100% | 20-60% | 30% |
| Harvest | | | |
| Tank volume | 10-99% | 10-90% | 80% |
| Harvest time | 2-10 days. | 5-10 days. | 9 days after start |
| Feed initiated | 6-0 gl⁻¹ | 3-0 gl⁻¹ | When glucose <2 gl⁻¹ |
| Repeated Batch | | | |
| Phases | | | |
| PH | 6-8 | 6.6-7.6 | 7.0 for CHO and 6.6-7.4 |
| | | | for BHK |
| Temperature | 28-40° C. | 30-37° C. | 37° C. for CHO and |
| | | | 36° C. for BHK |
| Temperature drop | | | |
| (OPTIONAL) | | | |
| Temperature drop to | 26-39° C. | 30-36° C. | 32° C. |
| Temperature drop at | 0.5-12.0 × 10⁶ cells | 0.5-12.0 × 10⁶ cells | 2.0-10 × 10⁶ cells ml⁻¹ |
| | ml⁻¹ | ml⁻¹ | |
| DOT | 10-100% | 20-60% | 30% |
| Harvest | | | |
| Tank volume | 10-99% | 10-90% | 80% |
| Harvest time | 1-7 days. | 1-7 days. | 5 days after harvest |
| Feed initiated | 3-0 gl⁻¹ | 3-0 gl⁻¹ | When glucose <2 gl⁻¹ |

### Fed-Batch Draw-Fill:

This process is a draw-Fill fermentation with a concentrated feed similar to the type proposed in the fed-batch process. A concern with a simple draw-fill process is that the fresh medium added may not be sufficient to sustain the cells over repeated batch fermentations. The inclusion of a feed would remove this worry. A feed would also allow operating the culture vessel with long batch times in a draw-fill process.

The composition (see Table 9) of the feed and the strategy for addition would be identical to that of the fed-batch process.

A process outline for this is shown in Table 11.

TABLE 11

| Setpoint | Range | Preferred range | More preferred Value |
|---|---|---|---|
| Initial Batch Phase | | | |
| PH | 6-8 | 6.6-7.6 | 7.0 for CHO and 6.6-7.4 |
| | | | for BHK |
| Temperature | 28-40° C. | 30-37° C. | 37° C. for CHO and |
| | | | 36° C. for BHK |
| Temperature drop | | | |
| (OPTIONAL) | | | |
| Temperature drop to | 26-39° C. | 30-36° C. | 32° C. |
| Temperature drop at | 0.5-12.0 × 10⁶ cells | 0.5-12.0 × 10⁶ cells | 2.0-10 × 10⁶ cells ml⁻¹ |
| | ml⁻¹ | ml⁻¹ | |
| DOT | 10-100% | 20-60% | 30% |
| Harvest | | | |
| Tank volume | 10-99% | 10-90% | 80% |
| Harvest time | 2-10 days | 5-10 days. | 9 days after start |
| Feed initiated | 6-0 gl⁻¹ | 3-0 gl⁻¹ | When glucose <2 gl⁻¹ |
| Repeated Batch | | | |
| Phases | | | |
| PH | 6-8 | 6.6-7.6 | 7.0 for CHO and 6.6-7.4 |
| | | | for BHK |
| Temperature | 28-40° C. | 30-37° C. | 37° C. for CHO and |
| | | | 36° C. for BHK |
| Temperature drop | | | |
| (OPTIONAL) | | | |
| Temperature drop to | 26-39° C. | 30-36° C. | 32° C. |
| Temperature drop at | 0.5-12.0 × 10⁶ cells | 0.5-12.0 × 10⁶ cells | 2.0-10 × 10⁶ cells ml⁻¹ |
| | ml⁻¹ | ml⁻¹ | |
| DOT | 10-100% | 20-60% | 30% |
| Harvest | | | |
| Tank volume | 10-99% | 10-90% | 80% |
| Harvest time | 1-7 days. | 1-7 days. | 5 days after harvest |
| Feed initiated | 6-0 gl⁻¹ | 3-0 gl⁻¹ | When glucose <2 gl⁻¹ |

### Sodium Butyrate Addition:

In one embodiment, the method of the present invention comprises the addition of sodium butyrate to the culture medium. Sodium butyrate has been shown to increase the production of recombinant proteins in a variety of cell types. This chemical is added at a specified concentration at a specific cell concentration in the culture vessel. It can be added during a batch or during on a regular basis in a perfusion process.

An outline of this aspect is shown in Table 12.

TABLE 12

| | | | More preferred |
|---|---|---|---|
| Setpoint | Range | Preferred range | Value |
| Sodium Butyrate | | 0.1-10 mM | 3 mM |
| Addition at | | 0.5-12.0 × 10⁶ cells | 2.0-10 × 10⁶ cells ml⁻¹ |
| | | ml⁻¹ | |

### Cells:

In practicing the present invention, the cells being cultivated are preferably mammalian cells, more preferably an established mammalian cell line, including, without limitation, CHO (e.g., ATCC CCL 61), COS-1 (e.g., ATCC CRL 1650), baby hamster kidney (BHK), and HEK293 (e.g., ATCC CRL 1573; Graham et al., J. Gen. Virol. 36:59-72, 1977) cell lines.

A preferred BHK cell line is the tk⁻ ts13 BHK cell line (Waechter and Baserga, Proc. Natl. Acad. Sci. USA 79:1106-1110, 1982), hereinafter referred to as BHK 570 cells. The BHK 570 cell line is available from the American Type Culture Collection, 12301 Parklawn Dr., Rockville, Md. 20852, under ATCC accession number CRL 10314. A tk⁻ ts13 BHK cell line is also available from the ATCC under accession number CRL 1632.

A preferred CHO cell line is the CHO K1 cell line available from ATCC under accession number CC161.

Other suitable cell lines include, without limitation, Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1); DUKX cells (CHO cell line) (Urlaub and Chasin, Proc. Natl. Acad. Sci. USA 77:4216-4220, 1980) (DUKX cells also being referred to as DXB11 cells), and DG44 (CHO cell line) (Cell, 33: 405, 1983, and Somatic Cell and Molecular Genetics 12: 555, 1986). Also useful are 3T3 cells, Namalwa cells, myelomas and fusions of myelomas with other cells. In some embodiments, the cells may be mutant or recombinant cells, such as, e.g., cells that express a qualitatively or quantitatively different spectrum of enzymes that catalyze post-translational modification of proteins (e.g., glycosylation enzymes such as glycosyl transferases and/or glycosidases, or processing enzymes such as propeptides) than the cell type from which they were derived.

In some embodiments, the cells used in practicing the invention are capable of growing in suspension cultures. As used herein, suspension-competent cells are those that can grow in suspension without making large, firm aggregates, i.e., cells that are monodisperse or grow in loose aggregates with only a few cells per aggregate. Suspension-competent cells include, without limitation, cells that grow in suspension without adaptation or manipulation (such as, e.g., hematopoietic cells or lymphoid cells) and cells that have been made suspension-competent by gradual adaptation of attachment-dependent cells (such as, e.g., epithelial or fibroblast cells) to suspension growth.

In some embodiments, the cells used in practicing the invention are adhesion cells (also known as anchorage-dependent or attachment-dependent cells). As used herein, adhesion cells are those that need to adhere or anchor themselves to a suitable surface for propagation and growth.

### Medium:

The present invention encompasses cultivating mammalian cells in medium lacking animal-derived components. As used herein, “animal-derived” components are any components that are produced in an intact animal (such as, e.g., proteins isolated and purified from serum) or are components produced by using components produced in an intact animal (such as, e.g., an amino acid made by using an enzyme isolated and purified from an animal to hydrolyse a plant source material).

By contrast, a protein which has the sequence of an animal protein (i.e., has a genomic origin in an animal) but which is produced in vitro in cell culture (such as, e.g., in a recombinant yeast or bacterial cell or in an established continuous mammalian cell line, recombinant or not), in media lacking components that are produced in, and isolated and purified from an intact animal is not an “animal-derived” component (such as, e.g., insulin produced in a yeast or a bacterial cell, or insulin produced in an established mammal cell line, such as, e.g., CHO, BHK or HEK cells, or interferon produced in Namalwa cells). For example, a protein which has the sequence of an animal protein (i.e., has a genomic origin in an animal) but which is produced in a recombinant cell in media lacking animal derived components (such as, e.g., insulin produced in a yeast or bacterial cell) is not an “animal-derived component. Accordingly, a cell culture medium lacking animal-derived components is one that may contain animal proteins that are recombinantly produced; such medium, however, does not contain, e.g., animal serum or proteins or other products purified from animal serum. Such medium may, for example, contain one or more components derived from plants.

Any cell culture medium lacking animal-derived components that supports cell growth and maintenanceunder the conditions of the invention may be used. Typically, the medium contains water, an osmolality regulator, a buffer, an energy source, amino acids, an inorganic or recombinant iron source, one or more synthetic or recombinant growth factors, vitamins, and cofactors. Media lacking animal-derived components and/or proteins are available from commercial suppliers, such as, for example, Sigma, JHR Biosciences, Gibco and Gemini.

In addition to conventional components, a medium suitable for producing Factor VII or a Factor VII-related polypeptide contains Vitamin K, which is required for γ-carboxylation of glutamic acid residues in Factor VII, at a concentration between about 0.1-50 mg/litre, preferably between about 0.5-25 mg/litre, more preferably between about 1-10 mg/litre and most preferably about 5 mg/litre.

In one embodiment, the medium used has the following composition:

The table below (Table 13) is a composition of a medium suitable for use in the present invention. Optionally, one or more of the components listed in Table 14 is/are added to the culture medium. Preferred ranges are listed in Table 14. In one embodiment, the medium used is Medium 318-X; in another embodiment, it is medium CHO-K.

TABLE 13

| | | Concentration in | Concentration in |
|---|---|---|---|
| COMPONENT | Range (mg/l) | CHO—K (mg/l) | 318-X (mg/l) |
| Sodium chloride | 0-70000 | 6122 | 6996 |
| Potassium chloride | 0-3118 | 311.8 | 311.8 |
| Sodium Dihydrogen Phosphate | 0-625 | 62.5 | 62.5 |
| monohydrate | | | |
| Sodium hydrogen carbonate | 0-27 | — | 2.7 |
| Disodium hydrogen | 0-710 | 71.02 | — |
| phosphate anhydrous | | | |
| Disodium hydrogen | 0-1340 | — | 134 |
| phosphate 7 hydrate | | | |
| Magnesium chloride anhydrous | 0-287 | 28.64 | — |
| Magnesium chloride 6 hydrate | 0-610 | — | 61 |
| Magnesium sulphate anhydrous | 0-488 | 48.84 | — |
| Magnesium sulphate 7 hydrate | 0-1000 | — | 100 |
| Calcium chloride anhydrous | 0-1166 | 116.6 | 116.6 |
| Copper sulphate 5 hydrate | 0-0.014 | 0.0013 | 0.0013 |
| Ferrous sulphate 7 hydrate | 0-4.17 | 0.147 | 0.417 |
| Ferric nitrate 9 hydrate | 0-0.5 | 0.05 | 0.05 |
| Ferric citrate | 0-123 | 0.4 | 12.24 |
| Zinc sulphate 7 hydrate | 0-0.44 | 0.432 | 0.432 |
| Dextrose anhydrous | 0-45000 | 4501 | 4500 |
| Linoleic acid | 0-12 | 1.189 | 0.336 |
| Insulin | 0-50 | 5 | 5 |
| DL 68 Thioctic Acid | 0-9 | 0.473 | 0.84 |
| l-alanine | 0-50 | 4.45 | 4.45 |
| l-arginine chloride | 0-5500 | 547.8 | 447.5 |
| l-asparagine monohydrate | 0-6010 | 407.5 | 607.5 |
| l-aspartic acid | 0-1100 | 6.65 | 106.65 |
| l-cysteine hydrochloride | 0-1200 | 117.65 | 77.56 |
| monohydrate | | | |
| l-glutamic acid | 0-2500 | 251.35 | 107.35 |
| Glycine | 0-190 | 18.75 | 18.75 |
| l-histidine hydrochloride | 0-2200 | 211.48 | 101.48 |
| monohydrate | | | |
| l-isoleucine | 0-750 | 54.47 | 74.47 |
| l-leucine | 0-1800 | 179.05 | 159.05 |
| l-lysine hydrochloride | 0-2400 | 231.25 | 131.25 |
| l-methionine | 0-1380 | 137.24 | 97.24 |
| l-phenylalanine | 0-1600 | 155.48 | 85.48 |
| l-proline | 0-1150 | 17.25 | 117.25 |
| l-serine | 0-4300 | 266.25 | 426.25 |
| l-threonine | 0-1800 | 173.45 | 73.45 |
| l-tryptophan | 0-2100 | 39.02 | 209.02 |
| l-tyrosine disodium | 0-900 | 55.79 | 85.79 |
| dihydrate | | | |
| l-valine | 0-1800 | 177.85 | 125.85 |
| l-cystine dihydrochloride | 0-320 | 31.29 | 31.29 |
| Sodium hypoxanthine | 0-25 | 2.39 | 2.39 |
| Putrescine dihydrochloride | 0-1 | 0.081 | 0.081 |
| Sodium pyruvate | 0-2300 | 220 | 55 |
| D-Biotin | 0-3 | 0.1313 | 0.259 |
| D-calcium pantothenate | 0-60 | 4.08 | 6 |
| Folic acid | 0-70 | 4.65 | 6.65 |
| I-inositol | 0-700 | 39.1 | 65.6 |
| Nicotinamide | 0-50 | 3.085 | 4.2 |
| Choline chloride | 0-450 | 29.32 | 42 |
| Pyridoxine hydrochloride | 0-25 | 0.117 | 2.2 |
| Riboflavin | 0-3 | 0.219 | 0.219 |
| Thiamine hydrochloride | 0-35 | 2.67 | 3.17 |
| Thymidine | 0-4 | 0.365 | 0.365 |
| Vitamin B12 | 0-50 | 2.68 | 4.68 |
| Pyridoxal hydrochloride | 0-60 | 6 | 2 |
| Glutathione | 0-50 | 2.5 | 5 |
| Sodium Selenite | 0-0.5 | 0.02175 | 0.0232 |
| l-ascorbic acid | 0-50 | 27.5 | 5 |
| Pluronic F68 | 0-10000 | 1000 | 1000 |
| Vitamin K | 0-50 | 5 | 5 |
| Dextran T 70 | 0-1000 | — | 100 |
| HY-SOY | 0-5000 | 500 | — |

### Optional Components:

TABLE 14

| Component | Range (mg/l) |
|---|---|
| Vegetable hydrolysates | 0-5000 |
| HyPep 4601, 4602, 4605, 5603, | |
| 7401 | |
| Lipids | 0-15 |
| Oleic acid | |
| Growth Factors | 0-50 |
| HGR, IGF, EGF | |

In another embodiment, the medium used has the following composition (318-U medium):

TABLE 15

| COMPONENT | |
|---|---|
| | MG/L |
| Sodium Chloride | 6122 |
| Potassium Chloride | 311.8 |
| Sodium Dihydrogen Phosphate Monohydrate | 62.5 |
| Disodium Hydrogen Phosphate Anhydrous | 71.02 |
| Magnesium Chloride Anhydrous | 28.64 |
| Magnesium Sulphate Anhydrous | 48.84 |
| Calcium Chloride Anhydrous | 116.6 |
| Copper Sulphate 5-hydrate | 0.0013 |
| Ferrous Sulphate 7-hydrate | 0.417 |
| Ferric Nitrate 9-hydrate | 0.05 |
| Zinc Sulphate 7-hydrate | 0.432 |
| Dextrose Anhydrous | 4501 |
| Linoleic Acid | 1.189 |
| DL-68-Thioctic Acid | 0.473 |
| L-Alanine | 4.45 |
| L-Arginine Hydrochloride | 547.5 |
| L-Asparagine Monohydrate | 407.5 |
| L-Aspartic Acid | 6.65 |
| L-Cysteine Hydrochloride Monohydrate | 117.65 |
| L-Glutamic Acid | 251.35 |
| L-Glutamine | 365 |
| Glycine | 18.75 |
| L-Histidine Hydrochloride Monohydrate | 211.48 |
| L-Isoleucine | 54.47 |
| L-Leucine | 179.05 |
| L-Lysine Hydrochloride | 231.25 |
| L-Methionine | 137.24 |
| L-Phenylalanine | 155.48 |
| L-Proline | 17.25 |
| L-Serine | 266.25 |
| L-Threonine | 173.45 |
| L-Tryptophan | 39.02 |
| L-Tyrosine Disodium Dihydrate | 55.79 |
| L-Valine | 177.85 |
| L-Cystine Dihydrochloride | 31.29 |
| Sodium Hypoxanthine | 2.39 |
| Putrescine Dihydrochloride | 0.081 |
| Sodium Pyruvate | 220 |
| D-Biotin | 0.1313 |
| D-Calcium Pantothenate | 4.08 |
| Folic Acid | 4.65 |
| I-Inositol | 39.1 |
| Nicotinamide | 3.085 |
| Choline Chloride | 29.32 |
| Pyridoxine Hydrochloride | 0.117 |
| Riboflavin | 0.219 |
| Thiamine Hydrochloride | 2.67 |
| Thymidine | 0.365 |
| Vitamin B12 | 2.68 |
| Pyridoxal Hydrochloride | 3 |
| Glutathione | 2.5 |
| Sodium Selenite | 0.02175 |
| L-Ascorbic Acid, Free Acid | 27.5 |
| Sodium Hydrogen Carbonate | 2440 |
| HySoy (soy protein hydrolysate) | 500 |
| Ethanolamin | 1.22 |
| Insulin | 5 |
| Dextran T70 | 100 |
| Pluronic F68 | 1000 |
| Vitamin K1 | 5 |
| | ML/L |
| Fe/citrat complex (50 mM/1 M) | 0.4 |
| Mercaptoethanol | 0.0035 |

In one embodiment, the medium is 318-X Medium and the cell line is a BHK cell line; in another embodiment, the medium is 318-U Medium and the cell line is a BHK cell line. In another embodiment, the medium is CHO-K Medium and the cell line is a CHO cell line.

In preferred embodiments, the cells used in practicing the present invention are adapted to suspension growth in medium lacking animal-derived components, such as, e.g., medium lacking serum. Such adaptation procedures are described, e.g., in Scharfenberg, et al., Animal Cell Technology Developments towards the 21^{st} Century, E. C. Beuvery et al. (Eds.), Kluwer Academic Publishers, pp. 619-623, 1995 (BHK and CHO cells); Cruz, Biotechnol. Tech. 11: 117-120, 1997 (insect cells); Keen, Cytotechnol. 17:203-211, 1995 (myeloma cells); Berg et al., Biotechniques 14:972-978, 1993 (human kidney 293 cells).

In a particularly preferred embodiment, the host cells are BHK 21 or CHO cells that have been engineered to express human Factor VII and that have been adapted to grow in the absence of serum or animal-derived components.

### Culture Methods

The present invention provides methods for large-scale cultivation of mammalian cells, which are carried out by the steps of:

(i) inoculating cells into a seed culture vessel containing culture medium lacking animal-derived components and propagating the seed culture at least until the cells reach a minimum cross-seeding density;

(ii) transferring the propagated seed culture to a large-scale culture vessel containing (a) culture medium lacking animal-derived components, under conditions in which the cells migrate onto the carriers (in case of a macroporous carrier process); and

(iii) propagating the large-scale culture in medium lacking animal-derived components, at least until said cells reach a useful density.

In some embodiments, the methods are carried out by the steps of:

(i) inoculating cells into a seed culture vessel containing culture medium lacking animal-derived components and propagating the seed culture at least until the cells reach a minimum cross-seeding density;

(ii) transferring the propagated seed culture to a large-scale culture vessel containing (a) culture medium lacking animal-derived components and (b) macroporous carriers, under conditions in which the cells migrate into the carriers; and

(iii) propagating the large-scale culture in medium lacking animal-derived components, at least until said cells reach a useful density.

In some embodiments, the methods further comprise the step of:

(iv) maintaining the culture obtained in step (iii) in medium lacking animal-derived components by regular harvesting of the culture medium and replacement by fresh medium.

The cooling step is done within 10-240 minutes, such as, e.g., 20-180 minutes, or 30-120 minutes, before sedimenting the cell-containing microcarriers. The step is typically carried out as follows: The bioreactor is cooled and the temperature is monitored. When the bioreactor reaches a pre-determined temperature below the setpoint temperature, such as, e.g., 10° C. below the set point of the culturing, the stirring of the bioreactor contents is stopped and the cell-containing carriers are sedimented. When media exchange has taken place, the temperature is again regulated to the setpoint of the culturing. The fresh media being added is typically pre-warmed to a temperature close to the setpoint of the cultivation.

Adhesion cells: In some embodiments of the invention, the process is a microcarrier process and the cells used are adhesion cells (attachment-dependent or anchorage-dependent cells). In these embodiments, both the propagation phase and the production phase include the use of microcarriers. The used adhesion cells should be able to migrate unto the carriers (and into the carriers if a macroporous carrier is used) during the propagation phase(s) and to migrate to new carrier when being transferred to the production bioreactor. If the adhesion cells are not sufficiently able to migrate to new carriers by themselves, they may be liberated from the carriers by contacting the cell-containing microcarriers with proteolytic enzymes or EDTA. The medium used (free of animal-derived components) should furthermore contain components suitable for supporting adhesion cells; suitable media for cultivation of adhesion cells are available from commercial suppliers, such as, e.g., Sigma.

If suspension-adapted or suspension-competent cells are used in a microcarrier process, the propagation of cells may be done in suspension, thus only in the production phase including the use of microcarriers.

Inoculation and initial propagation: It will be understood that step (i) may be repeated with a progressive increase in the size of the seed culture vessel, until a sufficient number of cells is obtained for step (ii). For example, one or more seed culture vessels of 5 l, 50 l, 1001 l, or 500 l may be used sequentially. A seed culture vessel as used herein is one that has a capacity of between about 5 l and 1000 l. Typically, cells are inoculated into a seed culture vessel at an initial density of about 0.2-0.4×10⁶ cells/ml and propagated until the culture reaches a cell density of about 1.0×10⁶ cells/ml. As used herein, a minimum cross-seeding density is between about 0.8 and about 1.5×10⁶ cells/ml.

Microcarriers: As used herein, microcarriers are particles, often cellulose- or dextran-based, which have the following properties:

(a) They are small enough to allow them to be used in suspension cultures (with a stirring rate that does not cause significant shear damage to cells);

(b) They are solid, porous, or have a solid core with a porous coating on the surface; and

(c) Their surfaces (exterior and interior surface in case of porous carriers) are positively charged.

In one series of embodiments, the microcarriers have an overall particle diameter between about 150 and 350 um; and have a positive charge density of between about 0.8 and 2.0 meq/g. Useful microcarriers include, without limitation, Cytodex 1™ and Cytodex 2™ (Amersham Pharmacia Biotech, Piscataway N.J.).

In one series of embodiments, the microcarrier is a solid carrier. Solid carriers are particularly suitable for adhesion cells (anchorage-dependent cells). In another series of embodiments, the microcarrier is a macroporous carrier.

Macroporous carriers: As used herein, macroporous carriers are particles, usually cellulose-based, which have the following properties: (a) They are small enough to allow them to be used in suspension cultures (with a stirring rate that does not cause significant shear damage to cells); (b) They have pores and interior spaces of sufficient size to allow cells to migrate into the interior spaces of the particle and (c) Their surfaces (exterior and interior) are positively charged. In one series of embodiments, the carriers:

(a) have an overall particle diameter between about 150 and 350 um;

(b) have pores having an average pore opening diameter of between about 15 and about 40 um; and

(c) have a positive charge density of between about 0.8 and 2.0 meq/g. In some embodiments, the positive charge is provided by DEAE (N. N,-diethylaminoethyl) groups. Useful macroporous carriers include, without limitation, Cytopore 1™ and Cytopore 2™ (Amersham Pharmacia Biotech, Piscataway N.J.). Particularly preferred are Cytopore 1 ™ carriers, which have a mean particle diameter of 230 um, an average pore size of 30 um, and a positive charge density of 1.1 meq/g.

Large-scale culture conditions: As used herein, a large-scale culture vessel has a capacity of at least about 100 l, preferably at least about 500 l, more preferably at least about 1000 l and most preferably at least about 5000 l. Typically, step (ii) involves transferring about 50 l of the propagated seed culture (having about 1.0×10⁶ cells/ml) into a 500 l culture vessel containing 150 l of culture medium. The large-scale culture is maintained under appropriate conditions of, e.g., temperature, pH, dissolved oxygen tension (DOT), O₂ and CO₂ tension, and agitation rate, and the volume is gradually increased by adding medium to the culture vessel.

In case of a microcarrier process the culture vessel also comprises about 750 g microcarriers. After the transfer, the cells typically migrate onto the surface of the carriers within the first 24 hours. In case of a macroporous carrier process the culture vessel also comprises about 750 g macroporous carriers. After the transfer, the cells typically migrate into the interior of the carriers within the first 24 hours.

The term “large-scale process” may be used interchangeably with the term “industrial-scale process”. Furthermore, the term “culture vessel” may be used interchangeably with “tank”, “reactor” and “bioreactor”.

High-level protein expression: When the cells are being propagated in order to produce high levels of a desired protein, the period of time until the cell density reaches a predetermined cell density (e.g., at least about 1×10⁶ cells/ml) is designated the “growth phase”. The growth phase normally comprises the steps (i), (ii) and (iii). When the cell density reaches the predetermined value (e.g., at least about 1×106 cells/ml, preferably at least 2×10⁶ cell/ml, more preferred 5×10⁶ cells/ml), the phase is designated the “production phase”. The production phase normally comprises step (iv). Any suitable, connected parameter changes are introduced at this stage.

### Culture Vessels:

The culture vessels may be e.g. conventional stirred tank reactors (CSTR) where agitation is obtained by means of conventional impeller types or airlift reactors where agitation is obtained by means of introducing air from the bottom of the vessel. Among the parameters controlled within specified limits are pH, dissolved oxygen tension (DOT), and temperature. The pH may be controlled by e.g. varying the CO2 concentration in the headspace gas and by addition of base to the culture liquid when required. Dissolved oxygen tension may be maintained by e.g. sparging with air or pure oxygen or mixtures thereof. The temperature-control medium is water, heated or cooled as necessary. The water may be passed through a jacket surrounding the vessel or through a piping coil immersed in the culture.

Once the medium has been removed from the culture vessel, it may be subjected to one or more processing steps to obtain the desired protein, including, without limitation, centrifugation or filtration to remove cells that were not immobilized in the carriers; affinity chromatography, hydrophobic interaction chromatography; ion-exchange chromatography; size exclusion chromatography; electrophoretic procedures (e.g. preparative isoelectric focusing (IEF), differential solubility (e.g., ammonium sulfate precipitation), or extraction and the like. See, generally, Scopes, Protein Purification, Springer-Verlag, New York, 1982; and Protein Purification, J.-C. Janson and Lars Ryden, editors, VCH Publishers, New York, 1989.

Purification of Factor VII or Factor VII-related polypeptides may involve, e.g., affinity chromatography on an anti-Factor VII antibody column (see, e.g., Wakabayashi et al., J. Biol. Chem. 261:11097, 1986; and Thim et al., Biochem. 27:7785, 1988) and activation by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat. No. 4,456,591; and Hedner et al., J. Clin. Invest. 71:1836 (1983). Alternatively, Factor VII may be activated by passing it through an ion-exchange chromatography column, such as Mono Q® (Pharmacia) or the like.

### Polypeptides for Large-Scale Production

In some embodiments, the cells used in practicing the invention are human cells expressing an endogenous Factor VII gene. In these cells, the endogenous gene may be intact or may have been modified in situ, or a sequence outside the Factor VII gene may have been modified in situ to alter the expression of the endogenous Factor VII gene.

In other embodiments, cells from any mammalian source are engineered to express human Factor VII from a recombinant gene. As used herein, “Factor VII” or “Factor VII polypeptide” encompasses wild-type Factor VII (i.e., a polypeptide having the amino acid sequence disclosed in U.S. Pat. No. 4,784,950), as well as variants of Factor VII exhibiting substantially the same or improved biological activity relative to wild-type Factor VII. The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa.

As used herein, “Factor VII-related polypeptides” encompasses polypeptides, including variants, in which the Factor VIIa biological activity has been substantially modified or reduced relative to the activity of wild-type Factor VIIa. These polypeptides include, without limitation, Factor VII or Factor VIIa into which specific amino acid sequence alterations have been introduced that modify or disrupt the bioactivity of the polypeptide.

The biological activity of Factor VIIa in blood clotting derives from its ability to (i) bind to tissue factor (TF) and (ii) catalyze the proteolytic cleavage of Factor IX or Factor X to produce activated Factor IX or X (Factor IXa or Xa, respectively). For purposes of the invention, Factor VIIa biological activity may be quantified by measuring the ability of a preparation to promote blood clotting using Factor VII-deficient plasma and thromboplastin, as described, e.g., in U.S. Pat. No. 5,997,864. In this assay, biological activity is expressed as the reduction in clotting time relative to a control sample and is converted to “Factor VII units” by comparison with a pooled human serum standard containing 1 unit/ml Factor VII activity. Alternatively, Factor VIIa biological activity may be quantified by (i) measuring the ability of Factor VIIa to produce of Factor Xa in a system comprising TF embedded in a lipid membrane and Factor X. (Persson et al., J. Biol. Chem. 272: 19919-19924, 1997); (ii) measuring Factor X hydrolysis in an aqueous system; (iii) measuring its physical binding to TF using an instrument based on surface plasmon resonance (Persson, FEBS Letts. 413: 359-363, 1997) and (iv) measuring hydrolysis of a synthetic substrate.

Factor VII variants having substantially the same or improved biological activity relative to wild-type Factor VIIa encompass those that exhibit at least about 25%, preferably at least about 50%, more preferably at least about 75% and most preferably at least about 90% of the specific activity of Factor VIIa that has been produced in the same cell type, when tested in one or more of a clotting assay, proteolysis assay, or TF binding assay as described above. Factor VII variants having substantially reduced biological activity relative to wild-type Factor VIIa are those that exhibit less than about 25%, preferably less than about 10%, more preferably less than about 5% and most preferably less than about 1% of the specific activity of wild-type Factor VIIa that has been produced in the same cell type when tested in one or more of a clotting assay, proteolysis assay, or TF binding assay as described above. Factor VII variants having a substantially modified biological activity relative to wild-type Factor VII include, without limitation, Factor VII variants that exhibit TF-independent Factor X proteolytic activity and those that bind TF but do not cleave Factor X.

Variants of Factor VII, whether exhibiting substantially the same or better bioactivity than wild-type Factor VII, or, alternatively, exhibiting substantially modified or reduced bioactivity relative to wild-type Factor VII, include, without limitation, polypeptides having an amino acid sequence that differs from the sequence of wild-type Factor VII by insertion, deletion, or substitution of one or more amino acids. Non-limiting examples of Factor VII variants having substantially the same biological activity as wild-type Factor VII include S52A-FVIIa, S60A-FVIIa (lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48: 501-505, 1995); and oxidized forms of Factor VIIa (Komfelt et al., Arch. Biochem. Biophys. 363: 43-54, 1999). Non-limiting examples of Factor VII variants having substantially reduced or modified biological activity relative to wild-type Factor VII include R152E-FVIIa (Wildgoose et al., Biochem 29: 3413-3420, 1990), S344A-FVIIa (Kazama et al., J. Biol. Chem. 270: 66-72, 1995), FFR-FVIIa (Holst et al., Eur. J. Vasc. Endovasc. Surg. 15: 515-520, 1998), and Factor VIIa lacking the Gla domain, (Nicolaisen et al., FEBS Letts. 317: 245-249, 1993).

The following examples are intended as non-limiting illustrations of the present invention.

### EXAMPLES

### Example 1

### Serum-Free Production of Factor VII

The following experiment was performed to produce Factor VII in large-scale culture.

A BHK cell line transfected with a Factor VII-encoding plasmid was adapted to growth in suspension culture in the absence of serum. The cells were adapted to serum-free suspension culture and were propagated sequentially in spinner cultures; as the cell number increased, the volume was gradually increased by addition of new medium.

Finally, 6 l of seed culture were inoculated into a 100-liter production bioreactor containing macroporous Cytopore 1 carriers (Pharmacia), after which the suspension cells became immobilized in the carriers within 24 hours after inoculation. The culture was maintained at 36° C. at a pH of 6.7-6.9 and a Dissolved Oxygen Tension (DOT) of 50% of saturation. The volume in the production bioreactor was gradually increased by addition of new medium as the cell number increased. When the cell density reached approximately 2×10⁶ cells/ml, the production phase was initiated and a medium change was performed every 24 hours: Agitation was stopped to allow for sedimentation of the cell-containing carriers, and 80% of the culture supernatant was then harvested and replaced with new medium. The harvested culture supernatant was filtered to remove non-trapped cells (i.e. cells that were not trapped in carriers) and cell debris and was then transferred for further processing.

During the production phase the cells reached 3-6×10⁶ cells/ml and a titer of 2-7 mg Factor VII/liter.

### Example 2

### Serum Free Production of Factor VII

The following experiment was performed to produce Factor VII in large-scale culture.

A plasmid vector pLN174 for expression of human FVII has been described (Persson and Nielsen. 1996. FEBS Lett. 385: 241-243). Briefly, it carries the cDNA nucleotide sequence encoding human FVII including the propeptide under the control of a mouse metallothionein promoter for transcription of the inserted cDNA, and mouse dihydrofolate reductase cDNA under the control of an SV40 early promoter for use as a selectable marker.

For construction of a plasmid vector encoding a gamma-carboxylation recognition sequence, a cloning vector pBluescript II KS+(Stratagene) containing cDNA encoding FVII including its propeptide was used (pLN171). (Persson et al. 1997. J. Biol. Chem. 272: 19919-19924). A nucleotide sequence encoding a stop codon was inserted into the cDNA encoding FVII after the propeptide of FVII by inverse PCR-mediated mutagenesis on this cloning vector. The template plasmid was denatured by treatment with NaOH followed by PCR with Pwo (Boehringer-Mannheim) and Taq (Perkin-Elmer) polymerases with the following primers:

5′-AGC GTT TTA GCG CCG GCG CCG GTG CAG GAC-3′
5′-CGC CGG CGC TAA AAC GCT TTC CTG GAG GAG CTG CGG
CC-3′

The resulting mix was digested with DpnI to digest residual template DNA and Escherichia coli were transformed with the PCR product. Clones were screened for the presence of the mutation by sequencing. The cDNA from a correct clone was transferred as a BamHI-EcoRI fragment to the expression plasmid pcDNA3 (Invitrogen). The resulting plasmid was termed pLN329. CHO K1 cells (ATCC CC162) were transfected with equal amounts of pLN174 and pLN329 with the Fugene6 method (Boehriner-Mannheim). Transfectants were selected by the addition of methotrexate to 1 μM and G-418 to 0.45 mg/ml. The pool of transfectants were cloned by limiting dilution and FVII expression from the clones was measured.

A high producing clone was further subcloned and a clone E11 with a specific FVII expression of 2.4 pg/cell/day in Dulbecco-modified Eagle's medium with 10% fetal calf serum was selected. The clone was adapted to serum free suspension culture in a commercially available CHO medium (JRH Bioscience) free of animal derived components.

The adapted cells were propagated sequentially in spinner cultures and as the cell number increased, the volume was gradually increased by addition of new medium.

After 25 days, 6 l of spinner culture were inoculated into a 50-liter bioreactor. The cells were propagated in the bioreactor and as the cell number increased, the volume was gradually increased by addition of new medium.

Finally, 50 l of seed culture were inoculated into a 500-liter production bioreactor containing macroporous Cytopore 1 carriers (Pharmacia), after which the suspension cells became immobilized in the carriers. The culture was maintained at 36° C. at a pH of 7.0-7.1 and a Dissolved Oxygen Tension (DOT) of 50% of saturation. The volume in the bioreactor was gradually increased by addition of new medium as the cell number increased. When the cell density reached approximately 10-12×10⁵ cells/ml, the production phase was initiated and a medium change was performed every 24 hours: agitation was stopped to allow for sedimentation of the cell-containing carriers, and 80% of the culture supernatant was then harvested and replaced with new medium. The harvested culture supernatant was filtered to remove non-trapped cells (i.e. cells that were not immobilized in carriers) and cell debris and was then transferred for further processing.

During the production phase the cells reached 2-3×10⁷ cells/ml and a titer of 8 mg factor VII/liter.

### Example 3

### Serum Free Production of Factor VII

The following experiment was performed to produce Factor VII in large-scale culture.

A high producing CHO clone was made as described in Example 2

The adapted cells were propagated sequentially in spinner cultures and as the cell number increased, the volume was gradually increased by addition of new medium.

After 25 days, 6 l of spinner culture were inoculated into a 50-liter bioreactor. The cells were propagated in the bioreactor and as the cell number increased, the volume was gradually increased by addition of new medium.

Finally, 50 l of seed culture were inoculated into a 500-liter production bioreactor containing macroporous Cytopore 1 carriers (Amersham Pharmacia Biotech), after which the suspension cells became immobilized in the carriers. The culture was maintained at 36° C. at a pH of 7.0-7.1 and a Dissolved Oxygen Tension (DOT) of 50% of saturation. The volume in the bioreactor was gradually increased by addition of new medium as the cell number increased. When the cell density reached approximately 10-12×10⁵ cells/ml, the production phase was initiated and a medium change was performed every 24 hours: agitation was stopped to allow for sedimentation of the cell-containing carriers, and 80% of the culture supernatant was then harvested and replaced with new medium. The harvested culture supernatant was filtered to remove non-trapped cells (i.e. cells that were not immobilized in carriers) and cell debris and was then transferred for further processing.

From day 14 onwards the medium was fortified with 2 g/l of HY—SOY (hydrolyzed soy protein).

From day 41 onwards cooling down of the culture to 10° C. below setpoint (i.e. to 26° C.) immediately before the daily medium exchange was introduced. The idea of this change was to reduce the oxygen requirements of the cells before the agitation is stopped and the carriers with cells are left to sediment at the bottom of the fermentor.

During the production phase the cells reached 2.5-3.5×10⁷ cells/ml and a titer of 8-13 mg factor VII/liter.

All patents, patent applications, and literature references referred to herein are hereby incorporated by reference in their entirety.

Many variations of the present invention will suggest themselves to those skilled in the art in light of the above detailed description. Such obvious variations are within the full intended scope of the appended claims.

1-20. (canceled)

21. A method for large-scale production of a Factor VII polypeptide in mammalian cells, comprising:
(i) propagating mammalian cells expressing a Factor VII polypeptide in suspension culture in a large-scale culture vessel containing medium lacking animal-derived components; and
(ii) during said propagating step, exchanging the medium continuously by a perfusion process.

22. A method as defined in claim 21, wherein 10-200% of the medium (by volume) is exchanged by perfusion over 10-48 hours of culture.

23. A method as defined in claim 22, wherein 80% of the medium (by volume) is exchanged by perfusion over 10-48 hours of culture.

24. A method as defined in claim 22, wherein 80% of the medium (by volume) is exchanged by perfusion over 24 hours of culture.

25. A method as defined in claim 21, wherein said cells, prior to said propagating step in a large-scale culture vessel, have been adapted to grow in medium lacking animal-derived proteins.

26. A method as defined in claim 21, wherein said cells, prior to said propagating step in a large-scale culture vessel, are capable of growing in suspension culture.

27. A method as defined in claim 21, wherein the mammalian cell is selected from the group consisting of BHK cells and CHO cells.

28. A method as defined in claim 21, wherein the mammalian cell is CHO-K1 cells.

29. A method as defined in claim 28, wherein the CHO-K1 cells are selected from the group consisting of cells designated CHO-K1, CHO-DXB 11, and CHO-DG44.

30. A method as defined in claim 21, wherein said Factor VII polypeptide is wild-type human Factor VII.

31. A method as defined in claim 21, wherein said Factor VII polypeptide is a Factor VII variant exhibiting at least about 25% increased biological activity relative to wild-type Factor VII produced in the same cell type under similar culture conditions.

32. A method as defined in claim 21, wherein said Factor VII polypeptide is produced at a level at least about 1 mg/l of culture.

33. A method as defined in claim 32, wherein said Factor VII polypeptide is produced at a level at least about 2.5 mg/l of culture.

34. A method as defined in claim 33, wherein said Factor VII polypeptide is produced at a level at least about 5 mg/l of culture.
